(19) United States
(12) Patent Application Publication
(10) Pub. No.: US 2026/0132167 A1
(43) Pub. Date: May 14, 2026

(54) METHODS OF INACTIVATING VIRAL CONTAMINANTS WITH A MIXTURE OF SOLVENT AND DETERGENT

(71) Applicant: IGI Therapeutics SA, La Chaux-de-Fonds (CH)

(72) Inventors: Laure CAHUZAC, La Chaux-de-Fonds (CH); Lionel DUARTE, La Chaux-de-Fonds (CH); Anais DURET

(73) Assignee: IGI Therapeutics SA, La Chaux-de-Fonds (CH)

(21) Appl. No.: 18/695,671

(22) PCT Filed: Sep. 29, 2022
(86) PCT No.: PCT/EP2022/077206

§ 371 Date: Mar. 26, 2024

(30) Foreign Application Priority Data
Sep. 30, 2021 (EP) 21200257.0

(51) Int. Cl. C07K 1/22; C07K 16/00

(52) U.S. Cl. CPC C07K 1/22; C07K 16/00; C07K 2317/31

(57) ABSTRACT

The present invention relates to methods of viral inactivation with a mixture of solvent and detergent. In particular, the present invention is directed to a method for preparing a virus-inactivated polypeptide solution wherein a polypeptide material is first loaded into an affinity chromatography column then incubated with said mixture of a solvent and a detergent within the affinity chromatography column.

### TECHNICAL FIELD

The present invention relates to methods of viral inactivation with a mixture of solvent and detergent. In particular, the present invention is directed to a method for preparing a virus-inactivated polypeptide solution wherein a polypeptide material is first loaded into an affinity chromatography column then incubated with said mixture of a solvent and a detergent within the affinity chromatography column.

### BACKGROUND

When biomolecules like proteins are used as pharmaceuticals for the treatment of diseases, the production process is designed to assure human health. Cell cultures and other biological materials, typically used for the production of therapeutic proteins, can be contaminated by viruses, either present in the source material or introduced during the production process. If not removed or irreversibly inactivated, the viral contaminates represent a risk to the health of the individuals using the drug. Therefore, to manufacture pharmaceuticals safe for use in humans, the produced proteins are required to be free of active viral contaminates (CPMP/ICH/295/95).

To produce therapeutic proteins, a purification process is carried out to isolate them from the rest of the starting mixed biological material. Steps of the purification, like chromatography and filtration, contribute to virus removal and are combined with specific viral inactivation techniques to assure viral clearance. Methods for virus inactivation are well known in the art. Inactivation is normally achieved by treatment with low pH (Shukla A A at al., Pharm. Bioprocess., 2015, 3(2): 127-138) or high temperatures (Pasteurization) (U.S. Pat. No. 4,749,783) that lead to viral proteins denaturation. These treatments are effective against viruses, but they may not be compatible with all therapeutic proteins, such as therapeutic antibodies, which may be susceptible to denaturation.

The complexity of the therapeutic protein my further set a challenge. For instance, a classical virus inactivation by low pH treatment may be not suitable for multispecific antibodies, such as bispecific antibodies, for which further stability challenges might be faced due to the complex structure of the molecule; an alternative to low pH treatment could be the use of solvents and detergents. Solvent and detergent (S/D) treatment is often used to assure viral inactivation in plasma products. In particular, the treatment of plasma products with a solvent/detergent mix impairs viral activity by solubilizing the virus lipid envelope, as established by the World Health Organization (WHO) Technical report, Annex 4 guidelines. Optimizations of this treatment have been carried out to allow S/D treatment of plasma product in a single-use bag system (Hsieh, Y T. et al. Transfusion. 2016 June; 56(6):1384-93); and to treat recombinant Factor VIII in lower temperature and shorter time conditions compared to the ones indicated by WHO Technical report, Annex 4 guidelines (WO2012082931). Additionally, in non-commercial, research scale S/D treatment has been applied as additional virus elimination step of an Anti-D antibody containing sample from human hybridoma cell line (Roberts P L, Biotechnol Prog. 2014 November-December; 30(6):1341-7).

Particularly challenging may be the solvent/detergent treatment of a cell harvest material that comprises a therapeutic antibody. In particular, the use of a S/D treatment in a clarified harvest may lead to several challenges at manufacturing scale. In fact, processing large volumes, e.g. 250 liters or more, of clarified harvest implies the use of large volumes of solvents which may be a carcinogenic, mutagenic and/or reprotoxic agent. Obtaining a virus inactivated material, while maintaining a high quality of the purified product, such as purified therapeutic antibody, from a clarified harvest remains a challenge in terms of maintenance of equipment required for the treatment, waste management and precautions for manipulation.

### SUMMARY

The classical virus inactivation by low pH treatment may not be suitable for multispecific antibodies, such as bispecific antibodies. An alternative may be viral inactivation by Solvent/Detergent (S/D) treatment of the clarified harvest, for instance before starting the purification process, e.g., by an affinity chromatography. However, S/D treatment of the clarified harvest may lead to several difficulties at manufacturing scale, given the necessity of using large volumes of solvent which may be a carcinogenic, mutagenic and reprotoxic. S/D treatment of the clarified harvest therefore represents a challenge in terms of equipment required for the treatment, waste management and precautions for manipulation.

We found herein an alternative to S/D incubation of the clarified harvest to reduce the volume of chemicals used and to solve the manufacturability challenges while demonstrating the efficiency of the process to remove the chemicals used. An identified option is to perform S/D treatment within the affinity chromatograph column, e.g., within a protein A (PA) chromatography column, in static mode, and thereby combine the affinity chromatograph and S/D treatment in a single step.

The present invention relates to a method for preparing a virus-inactivated polypeptide solution, comprising the steps of (i) harvesting a polypeptide material produced by transfected cells comprising the coding sequences of said polypeptide, which have undergone cell culture, (ii) viral inactivation treatment upon said harvested polypeptide material consisting of incubation with a mixture of a solvent and a detergent, characterized in that said harvested polypeptide material is first loaded into an affinity chromatography column that has been equilibrated with an equilibration buffer and then incubated with said mixture of a solvent and a detergent within said affinity chromatography column.

In particular embodiments, the affinity chromatography column is a Protein A affinity chromatography column.

In particular embodiments, the incubation of said harvested polypeptide material with said mixture of a solvent and a detergent is performed at a pH comprised between about 6.5 and about 8.5.

In certain specific embodiments, the incubation of said harvested polypeptide material with said mixture of a solvent and a detergent is performed at a pH comprised between about 7 and about 8.

In a particular embodiment the incubation of said harvested polypeptide material with said mixture of a solvent and a detergent is performed at a pH of about 7.4.

In another particular embodiment the incubation of said harvested polypeptide material with said mixture of a solvent and a detergent is performed at a pH of about 8.

In particular embodiments, the incubation of said harvested polypeptide material with said mixture of a solvent and a detergent is preceded by at least one washing step.

In particular embodiments, the incubation of said harvested polypeptide material with said mixture of a solvent and a detergent is followed by at least one washing step.

In other particular embodiments, the incubation of said harvested polypeptide material with said mixture of a solvent and a detergent is followed by two washing steps.

In particular embodiments, the polypeptide is eluted from said affinity chromatography column with an elution buffer.

In particular embodiments, said solvent is TnBP and said detergent is Polysorbate 80.

In more particular embodiments, the concentration of TnBP is comprised between about 0.05% (w/w) and about 1% (w/w), and the concentration of Polysorbate 80 is comprised between about 0.5% (w/w) and about 2% (w/w).

In certain specific embodiments, the mixture of a solvent and a detergent comprises about 0.3% (w/w) TnBP and about 1% (w/w) Polysorbate 80.

In certain embodiments, the washing step that precedes the incubation of said polypeptide with said mixture of a solvent and a detergent is performed with a washing buffer comprising about 0.1 M of Tris buffer and 1 M NaCl at pH 8.0 and 1% (w/w) Polysorbate 80 and TnBP 0.3% (w/w).

In certain embodiments, the at least one washing step that follows the incubation of said polypeptide with said mixture of a solvent and a detergent is performed with a washing buffer that comprises about 0.17 M of Acetate at pH 5.0.

In other particular embodiments, the incubation of said harvested polypeptide material with said mixture of a solvent and a detergent is followed by two washing steps wherein the first of said two washing steps that follows the incubation of said polypeptide with said mixture of a solvent and a detergent is performed with a washing buffer comprising about 0.1 M of Tris buffer and 1 M NaCl at pH 8.0, and the second of said two washing steps that follows the incubation of said polypeptide with said mixture of a solvent and a detergent is performed with a washing buffer comprising about 0.17 M of Acetate at pH 5.0.

In a specific embodiment, the method according to the present invention comprises the steps of:
(a) Equilibrating a protein A affinity chromatography column with an equilibration buffer comprising PBS at pH 7.4;
(b) Loading a polypeptide material onto said Protein A affinity chromatography column material at a loading factor 25 g/L;
(c) Incubating said polypeptide material with a mixture of a solvent and a detergent that comprises about 0.3% (w/w) TnBP and about 1% (w/w) Polysorbate 80 for about 60 minutes at pH 7.4;
(d) Subjecting said protein A affinity chromatography column to two washing steps, wherein the first of the two washing steps is performed with a washing buffer comprising about 0.1 M of Tris buffer and 1 M NaCl at pH 8.0, and the second of said two washing steps t is performed with a washing buffer comprising about 0.17 M of Acetate at pH 5.0;
(e) Eluting said polypeptide with an elution buffer comprising about 0.05 M Acetate at pH 4.1.

In certain embodiments, the polypeptide according to the present invention is an antibody or an antibody fragment thereof.

More specifically, the antibody or an antibody fragment thereof is a monoclonal antibody. Even more specifically, the antibody or an antibody fragment thereof is a recombinant antibody.

In certain embodiments, the antibody according to the present invention is multispecific; preferably bispecific.

In certain embodiments, said harvested polypeptide material is produced in non-human mammalian cells.

As utilized in accordance with the present disclosure, the following terms, unless otherwise indicated, shall be understood to have the following meanings. Unless otherwise required by context, singular terms shall include pluralities and plural terms shall include the singular. As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that aspects and embodiments of the present disclosure described herein include “comprising,” “consisting,” and “consisting essentially of aspects and embodiments.

In the present invention, the term “antibody” and the term “immunoglobulin” are used interchangeably. The term “antibody” as referred to herein, includes the full-length antibody and antibody fragments. Antibodies are glycoproteins produced by plasma cells that play a role in the immune response by recognizing and inactivating antigen molecules. In mammals, five classes of immunoglobulins are produced: IgM, IgD, IgG, IgA and IgE. In the native form, immunoglobulins exist as one or more copies of a Y-shaped unit composed of four polypeptide chains: two identical heavy (H) chains and two identical light (L) chains. Covalent disulfide bonds and non-covalent interactions allow inter-chain connections; particularly heavy chains are linked to each other, while each light chain pairs with a heavy chain. Both heavy chain and light chain comprise an N-terminal variable (V) region and a C-terminal constant (C) region. In the heavy chain, the variable region is composed of one variable domain (VH), and the constant region is composed of three or four constant domains (CH1, CH2, CH3 and CH4), depending on the antibody class; while the light chain comprises a variable domain (VL) and a single constant domain (CL). The variable regions contain three regions of hypervariability, termed complementarity determining regions (CDRs). These form the antigen binding site and confer specificity to the antibody. CDRs are situated between four more conserved regions, termed framework regions (FRs) that define the position of the CDRs. Antigen binding is facilitated by flexibility of the domains position; for instance, immunoglobulin containing three constant heavy domains present a spacer between CH1 and CH2, called “hinge region” that allows movement for the interaction with the target. Starting from an antibody in its intact, native form, enzymatic digestion can lead to the generation of antibody fragments. For example, the incubation of an IgG with the endopeptidase papain, leads to the disruption of peptide bonds in the hinge region and to the consequent production of three fragments: two antibody binding (Fab) fragments, each capable of antigen binding, and a cristallizable fragment (Fc). Digestion by pepsin instead yields one large fragment, F(ab′)2, composed by two Fab units linked by disulfide bonds, and many small fragments resulting from the degradation of the Fc region. Depending on their nature, antibodies and antibody fragments can be monomeric or multimeric, monovalent or multivalent, monospecific or multispecific.

The term “full-length antibody” as used herein, includes antibodies in their native intact structure that comprises at least two pairs of heavy and light chains.

The term “antibody fragments” as used herein, includes one or more portion(s) of a full-length antibody. Non limiting examples of antibody fragments include: (i) the fragment crystallizable (Fc) composed by two constant heavy chain fragments which consist of CH2 and CH3 domains, in IgA, IgD and IgG, and of CH2, CH3 and CH4 domains, in IgE and IgM, and which are paired by disulfide bonds and non-covalent interactions; (ii) the fragment antigen binding (Fab), consisting of VL, CL and VH, CH1 connected by disulfide bonds; (iii) Fab′, consisting of VL, CL and VH, CH1 connected by disulfide bonds, and of one or more cysteine residues from the hinge region; (iv) Fab′-SH, which is a Fab′ fragment in which the cysteine residues contain a free sulfhydryl group; (v) F(ab′)2 consisting of two Fab fragments connected at the hinge region by a disulfides bond; (vi) the variable fragments (Fv), consisting of VL and VH chains, paired together by non-covalent interactions; (vii) the single chain variable fragments (scFv), consisting of VL and VH chains paired together by a linker; (ix) the bispecific single chain Fv dimers, (x) the scFv-Fc fragment; (xi) a Fd fragment consisting of the VH and CH1 domains; (xii) the single domain antibody, dAb, consisting of a VH domain or a VL domain; (xiii) diabodies, consisting of two scFv fragments in which VH and VL domains are connected by a short peptide that prevent their pairing in the same chain and allows the non-covalent dimerization of the two scFvs; (xiv) the trivalent 10 triabodies, where three scFv, with VH and VL domains connected by a short peptide, form a trimer; (xv) half-IgG, comprising a single heavy chain and a single variable chain; (xvi) an antibody chain or a portion of it (e.g. an antibody heavy chain or a light chain or a portion of it).

The term “homo-dimeric antibody” or “homo-dimeric fragment” or “homo-dimer” as used herein includes an immunoglobulin molecule or part of comprising at least a first and a second polypeptide, like a first and a second domain, wherein the second polypeptide is identical in amino acid sequence to the first polypeptide. Preferably, a homo-dimeric immunoglobulin comprises two polypeptide chains, wherein the first chain has at least one identical domain to the second chain, and wherein both chains assemble, i.e., interact through their identical domains. Specifically, a homo-dimeric immunoglobulin comprises at least two identical domains and wherein both domains assemble, i.e., interact through their protein-protein interfaces. Preferably, a homo-dimeric immunoglobulin fragment comprises at least two domains, wherein the first domain is identical to the second domain, and wherein both domains assemble, i.e., interact through their protein-protein interfaces.

The term “hetero-dimeric antibody” or “hetero-dimeric fragment” or “hetero-dimer” as used herein includes an immunoglobulin molecule or part of comprising at least a first and a second polypeptide, like a first and a second domain, wherein the second polypeptide differs in amino acid sequence from the first polypeptide. Preferably, a hetero-dimeric immunoglobulin comprises two polypeptide chains, wherein the first chain has at least one non identical domain to the second chain, and wherein both chains assemble, i.e., interact through their non-identical domains. Specifically, a hetero-dimeric immunoglobulin comprises at least two domains, wherein the first domain is non identical to the second domain, and wherein both domains assemble, i.e., interact through their protein-protein interfaces. More preferably the hetero-dimeric immunoglobulin, has binding specificity for at least two different ligands, antigens, or binding sites, i.e., is bispecific.

The term “valence” as used herein, refers to the number of binding sites in the antibody. An antibody that has more than one valence is called multivalent; non-limiting examples of multivalent antibodies are: bivalent antibody, characterized by two biding sites, trivalent antibody, characterized by three binding sites, and tetravalent antibody, characterized by four binding sites.

The term “monospecific antibody” as used herein, refers to any antibody or fragment having one or more binding sites, all binding the same epitope.

The term “multispecific antibody” as used herein, refers to any antibody or fragment having more than one binding site that can bind different epitopes of the same antigen, or different antigens. A non-limiting example of multispecific antibodies are bispecific antibody.

The term “bispecific antibody” refers to any antibody having two binding sites that can bind two different epitopes of the same antigen, or two different antigens.

The term “antigen” as used herein, refers to any molecule to which an antibody can specifically bind. Examples of antigens include polypeptides, proteins, polysaccharides and lipid molecules. In the antigen one or more epitopes can be present. The term “epitope” or “antigenic determinant” as used herein, refers to the portion of the antigen that makes the direct chemical interaction with the antibody.

The term “monoclonal antibody” as used herein, refers to antibodies that are produced by clone cells all deriving from the same single cell, and that specifically bind the same epitope of the target antigen. When therapeutic antibodies are produced, the generation of monoclonal antibodies is preferred over polyclonal antibodies. In fact, while monoclonal antibodies are produced by cells originating from a single clone and bind all the same epitope, polyclonal antibodies are produced by different immune cells and recognize multiple epitopes of a certain antigen. Monoclonal antibodies assure batch to batch homogeneity, reduced cross-reactivity and high specificity toward the target. Monoclonal antibodies can be expressed, for instance in host cells, using recombinant DNA, giving rise to a recombinant antibody.

The term “recombinant antibody” as used herein, refers to an antibody that has been produced by any process involving the use of recombinant DNA. A recombinant antibody can be engineered in such a way to improve characteristics such as immunogenicity, binding affinity, molecular size, specificity, half-life, and format. Examples of recombinant antibodies include, but are not limited to engineered antibodies, chimeric antibodies, CDRs grafted antibodies (such as humanized antibodies), fully human antibodies, antibody fragments, Fc-engineered antibodies, multispecific antibody (such as bispecific, trispecific, tetraspecific antibody), monomeric and multimeric antibodies (such as homo-dimeric and hetero-dimeric antibodies).

The term “CDRs grafted antibody” as used herein, refers to an antibody in which CDRs derived from one species are grafted in the framework region of another species; a non-limiting example of CDRs grafted antibody is a humanized antibody in which CDRs from a mammalian species, such as mouse, are grafted in a human framework region.

The term “cell transfection” refers to the introduction of foreign genetic material (e.g., a vector) into a host cell, such as a eukaryotic cell. The term “transfected cell” refers to a cell wherein foreign genetic material has been introduced. When the protein codified by the artificially introduced nucleic acid is expressed by the cells, it provides the genetically modified cells with properties different than the respective wild type form. The introduced nucleic acid can be DNA or RNA. Examples of techniques commonly used for introducing exogenous nucleic acid into the host cells include chemical-based methods, where the transfection is mediated by transfection reagents such calcium-phosphate, liposomes, cationic polymers or dendrimers; physical-based method such as electroporation and microinjection; and virus-based methods where virus infection mediates gene delivery. Using these techniques, transient or stable transfection can be achieved. In the transient transfection the nucleic acid sequence does not integrate into the genome of the host cell, therefore the expression of the protein codified by the exogenous genetic material is limited in time, while stable transfection is achieved when the cells integrate the foreign genetic material in their genome, giving rise to a stable transfected cell line.

The term “vector” as used herein refers to any molecule (e.g., nucleic acid, plasmid, or virus) that is used to transfer coding information to a host cell. The term “vector” includes a nucleic acid molecule that is capable of transporting another nucleic acid to which it has been linked. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms “plasmid” and “vector” may be used interchangeably herein, as a plasmid is the most commonly used form of vector. However, the disclosure is intended to include other forms of expression vectors, such as viral vectors (e.g., replication defective retroviruses, adenoviruses, and adeno-associated viruses), which serve equivalent functions.

The phrase “recombinant host cell” (or “host cell”) as used herein refers to a cell into which a recombinant expression vector has been introduced, e.g., a transfected cell. A recombinant host cell or host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the polypeptide of interest, including bacterial, yeast, baculoviral, and mammalian expression systems (as well as phage display expression systems). To express the polypeptide of interest recombinantly, a host cell is transformed or transfected with one or more recombinant expression vectors carrying DNA fragments encoding the polypeptide such that the polypeptides are expressed in the host cell and, preferably, secreted into the medium in which the host cells are cultured, from which medium the binding protein can be recovered.

More in particular, the term “host cells” refers to all the cells in which the protein codified by the transfected nucleic acid material is expressed, including those cells in which the foreign nucleic acid is directly introduced and their progeny. Cell lines suitable for the expression of the polypeptide of the present invention, such as an antibody include and are not limited to bacteria, mammalian, insect, plant and yeast cells. Cell lines often used for the expression and production of therapeutic antibodies are mammalian cells lines such as Chinese hamster ovary (CHO) cells, NSO mouse myeloma cells, human cervical carcinoma (HeLa) cells and human embryonic kidney (HEK) cells. Host cells are cultured in conditions that aid their growth and the expression of the polypeptide, such as of the antibody or antibody chains.

Cell culture is a process cells are grown under controlled conditions in an artificial environment. The terms “cell culture” and “culture” refer to the growth and/or propagation and/or maintenance of cells in controlled artificial conditions. Optimal culturing conditions are obtained by the control and adjustment of several parameters including: the formulation of the cell culture medium, the bioreactor operating parameters, the nutrient supply modality and the culturing time period. The formulation of the culturing medium has to be optimized to favorite cell vitality and reproduction; examples of constituents of the cell culture medium include but are not limited to essential amino acids, salts, glucose, growth factors and antibiotics. Important bioreactor operating parameters are: temperature, pH, agitation speed, oxygenation and carbon dioxide levels. Nutrients can be supplied in different ways: in the batch mode culture all the necessary nutrients are present in the initial base medium and are used till exhausted while wastes accumulate; in the fed-batch culture additional feed medium is supplied to prevent nutrient depletion and prolong the culture; differently, in the perfusion modality, cells in culture are continuously supplemented with fresh medium containing nutrients that flows in the bioreactor removing cell wastes. The culturing period is important as it needs to be long enough to let the cells produce a consistent amount of product, but it cannot be too long to impair cell viability.

The term “harvested polypeptide material”, refers to the material, obtained by the cell culture, containing a polypeptide expressed by the host cells, for instance the antibody expressed by the host cells. The harvested polypeptide material may be produced by first harvesting the host cell culture and then subjecting the harvest to a process of clarification which allows the removal of cell debris through steps of centrifugations and/or filtrations. In particular, clarification comprises one or more steps that aid the removal of cells, cell debris and colloidal particles, to obtained clarified cell culture, comprising cell culture medium and the polypeptide.

The term “viral clearance” refers to any treatment that effectively remove and/or inactivate viruses which could contaminate the material of interest. When applied in the purification process of a polypeptide such as a therapeutic antibody, virus clearance refers to any method that leads to viral inactivation or viral removal from an antibody-containing material.

The term “viral inactivation” refers to any treatment that makes a virus unable to infect biological samples or to replicate. Viral inactivation can be achieved by different techniques such as the incubation of the biological sample with solvents and detergents (S/D), which causes viral inactivation trough the solubilization of the viral envelope; the incubation in low or high pH, which leads to the denaturation of the viral proteins; the pasteurization treatment, in which viral protein denaturation is achieved by high temperatures. The results of method comprising a step of viral inactivation treatment upon a polypeptide material, such as a harvested polypeptide material, is a virus-inactivated polypeptide solution. In certain embodiments, the result of the method of this invention is a virus-inactivated polypeptide solution wherein the polypeptide is an antibody or antibody fragment thereof.

The term “incubation” refers to the operation of keeping a material in certain conditions, comprising conditions for which the material undergoes modifications. In the process of viral inactivation, the incubation of a material with a solution having chemical characteristics that cause viral inactivation, leads to the generation of a material free of active viral contaminants, e.g., to a virus-inactivated polypeptide solution.

The term “solvent detergent treatment” refers to the incubation of the harvested antibody material with a solvent, such as an organic solvent or with an organic solvent and a detergent.

The term “virus removal” refers to any treatment that allows the physical separation of the viral particles from the treated sample. In the process of therapeutic protein purification, virus removal is accomplished by filtration steps. Additionally other phases of the purification process, such as chromatography steps, aid the removal of viral particles.

The term “filtration” refers to the operation of separating the solids from a fluid.

The term “chromatography” refers to the operation of separating compounds of a mixture based on their capability to interact with a stationary phase of a chromatography resin, from which they can be retained or eluted. Chromatography techniques are known in the art, for instance ion exchange chromatography separates ions and polar molecules based on their difference of charges, example of ion exchange chromatography techniques are cation exchange chromatography and anion exchange chromatography. Affinity chromatography relies on the specific interaction of the protein with an immobilized ligand. Non limiting examples of ligands which are useful for purification of an antibody by affinity chromatography are Protein A and Protein G. Protein A is a cell wall protein isolated from Staphylococcus aureus, which has the property of binding to the immunoglobulin Fc (and not binding to the antigen binding site). Herein, the term “Protein A chromatography” refers to an affinity chromatography wherein ligand binding the antibody is Protein A, wherein the term “Protein A” includes native Protein A, recombinant Protein A, and analogs or derivatives thereof Both protein analogs and derivatives retain their binding activity to antibodies (e.g., IgG Fc). Protein G is a cell wall protein isolated from type G Streptococcus, and its N-terminal part is albumin binding domain, and its C-terminal part is IgG binding domain and cell wall binding domain. Herein, the term “Protein G chromatography” refers to an affinity chromatography wherein ligand binding the antibody is Protein G, wherein the term “protein G” includes native protein G, recombinant protein G, and analogs or derivatives thereof. Both protein analogs and derivatives retain their binding activity to antibodies (e.g., IgG Fc).

The terms “viral inactivation assay” and “viral removal validation (VRV)” as used herein are interchangeable and refer to a procedure for testing the effective virus reduction. To perform viral inactivation assays, the viral presence in the samples is measured before and after spiking with a known quantity of stock virus and by comparing the output viral titre against the respective load viral titre. A log₁₀ reduction factor (RF) may be then determined according to the following formula: RF=log₁₀ {Input virus titre X Input volume/Output virus titre X Output volume. The inactivation/removal of viruses can be described as: “effective”, when RF is greater than 4 log₁₀; “moderately effective”, when RF is included between 2 log₁₀ and 4 log₁₀; “contributing to virus reduction”, RF is included between 1 log₁₀ and 2 log₁₀; and “ineffective”, when RF is less than 1 log₁₀.

In the present invention, the harvested polypeptide material, is a harvested antibody material, and comprises a monoclonal antibody. In particular embodiments, the polypeptide of the present invention is an antibody or an antibody fragment, or antibody chain thereof. Preferably a monoclonal antibody, which is recombinant. In a specific embodiment of the present invention, the monoclonal recombinant antibody is hetero-dimeric. In other specific embodiments of the present invention, the antibody is multispecific and/or multivalent. In a particular embodiment of the antibody of the present invention is a bispecific antibody, e.g., a hetero-dimeric bispecific antibody.

In the present invention, the hetero-dimeric bispecific antibody may be generated by BEAT® technology (WO2012131555).

The antibody of the present invention is expressed in host cells upon cell transfection. Cell transfection methods adopted in this invention include but are not limited to chemical-based methods exploiting a transfection reagent. Transfection reagents suitable for this invention include but are not limited to calcium-phosphate, liposome, cationic polymers and dendrimers. In one embodiment of this invention, cell transfection is carried out by a cationic polymer. Non limiting examples of a cationic polymer are diethylethanolamine and polyethylenimine. In a specific embodiment of this invention, the cation polymer is polyethylenimine.

In accordance with a particular aspect of the present invention, the host cell lines utilized for antibody production include but are not limited to eukaryotic cell lines. In one embodiment of this invention, the host cell lines are mammalian cell lines. In a more specific embodiment, the mammalian cell line is a non-human cell line. In an even more specific embodiment of this invention, the mammalian cell line is CHO cell line, e.g., CHO-S cell line.

In the present invention the virus-inactivated antibody solution may be prepared starting from a harvested antibody material. Particularly, the harvested antibody material is the product of the clarification of the bulk harvest of the host cell culture. In a specific embodiment of the present invention, the harvested antibody material is prepared by clarifying the bulk harvest through filtration steps, for instance including “dead end” depth filtration, followed by aseptically filtration through a 0.2 μm filter.

In a preferred embodiment of this invention, viral inactivation is carried out by solvent detergent treatment. Solvents useful for the method disclosed herein are organic solvents. Non limiting examples of organic solvents useful in the method of the present application include alcohol and alkylphosphate, e.g., dialkylphosphates and trialkylphosphate such as tri-(n-butyl)phosphate, tri-(t-butyl)phosphate, tri-(n-hexyl)phosphate, tri-(2-ethylhexyl)phosphate, or tri-(n-decyl)phosphate. In a preferred embodiment of this invention the solvent is tri-(n-butyl)phosphate (TnBP). Mixtures of different solvents can also be employed to carried out the present invention. The concentration of the organic solvent may be equal to or greater than about 0.1% (w/w) and equal to or less than about 1% (w/w). In particular the concentration of the organic solvent is selected from the group consisting of: about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w) or about 1% (w/w). More in particular, the concentration the organic solvent is at least about 0.1% (w/w), at least about 0.3% (w/w), at least about 0.5% (w/w), or at least about 1% (w/w). In certain preferred embodiments, the concentration of the organic solvent is equal to or greater than about 0.2% (w/w) and equal to or less than about 0.4% (w/w). In particular, the concentration of the organic solvent is about 0.2% (w/w), or about 0.3% (w/w), or about 0.4% (w/w). More preferably the concentration of the organic solvent is about 0.3% (w/w). The present invention also includes the concentrations of the organic solvent at intervals of 0.1% (w/w), 0.2% (w/w), 0.3% (w/w), 0.4% (w/w), 0.5% (w/w), 0.6% (w/w), 0.7% (w/w), 0.8% (w/w), 0.9% (w/w) or 1% (w/w) between the above cited concentrations.

Detergents useful in the method disclosed herein include ionic surfactants, zwitterionic (amphoteric) surfactants, non-ionic surfactants, or any combination therein. Ionic surfactants include anionic surfactants based on permanent (sulfate, sulfonate, phosphate) or pH dependent (carboxylate) anions. Anionic surfactants include, without limitation, alkyl sulfates like ammonium lauryl sulfate and sodium lauryl sulfate (SDS); alkyl ether sulfates like sodium laureth sulfate and sodium myreth sulfate; docusates like dioctyl sodium sulfosuccinate; sulfonate fluorosurfactants like perfluorooctanesulfonate (PFOS) and perfluorobutanesulfonate; alkyl benzene sulfonates; alkyl aryl ether phosphates; alkyl ether phosphates; alkyl carboxylates like fatty acid salts and sodium stearate; sodium lauroyl sarcosinate; and carboxylate fluorosurfactants like perfluorononanoate and perfluorooctanoate. Ionic surfactants also include cationic surfactants based on permanent or pH dependent cations. Cationic surfactants include, without limitation, alkyltrimethylammonium salts like cetyl trimethylammonium bromide (CTAB) and cetyl trimethylammonium chloride (CTAC); cetylpyridinium chloride (CPC); polyethoxylated tallow amine (POEA); benzalkonium chloride (BAC); benzethonium chloride (BZT); 5-Bromo-5-nitro-1,3-dioxane; dimethyldioctadecylammonium chloride; and dioctadecyldimethylammonium bromide (DODAB), as well as pH-dependent primary, secondary or tertiary amines like surfactants where the primary amines become positively charged at pH<10, or the secondary amines become charged at pH<4, like octenidine dihydrochloride. Zwitterionic surfactants are based on primary, secondary or tertiary amines or quaternary ammonium cation with a sulfonate, a carboxylate, or a phosphate. Zwitterionic surfactants include, without limitation, 3-[(3-Cholamidopropyl)dimethylammonio]-1-propanesulfonate (CHAPS); sultaines like cocamidopropyl hydroxysultaine; betaines like cocamidopropyl betaine; or lecithins. Non-ionic surfactants are less denaturing and as such are useful to solubilize membrane proteins and lipids while retaining protein-protein interactions. Non-limiting examples of surfactants include polyoxyethylene glycol sorbitan alkyl esters like polysorbate 20 sorbitan monooleate (such as TWEEN® 20), polysorbate 40 sorbitan monooleate (such as TWEEN® 40), polysorbate 60 sorbitan monooleate (such as TWEEN® 60), polysorbate 61 sorbitan monooleate (such as TWEEN® 61), polysorbate 65 sorbitan monooleate (such as TWEEN® 65), polysorbate 80 sorbitan monooleate (such as TWEEN® 80), and polysorbate 81 sorbitan monooleate (such as TWEEN® 81); poloxamers (polyethylene-polypropylene copolymers), like Poloxamer 124 (such as PLURONIC® L44), Poloxamer 181 (such as PLURONIC® L61), Poloxamer 182 (such as PLURONIC® L62), Poloxamer 184 (such as PLURONIC® L64), Poloxamer 188 (such as PLURONIC® F68), Poloxamer 237 (such as PLURONIC® F87), Poloxamer 338 (such as PLURONIC® L108), Poloxamer 407 (such as PLURONIC® F127); alkyl phenol polyglycol ethers; polyethylene glycol alkyl aryl ethers; polyoxyethylene glycol alkyl ethers, like octaethylene glycol monododecyl ether, pentaethylene glycol monododecyl ether, BRIJ® 30, and BRIJ® 35; 2-dodecoxyethanol (such as LUBROL®-PX); polyoxyethylene glycol octylphenol ethers like polyoxyethylene (4-5) p-t-octyl phenol (such as TRITON® X-45) and polyoxyethylene octyl phenyl ether (such as TRITON® X-100); polyoxyethylene glycol alkylphenol ethers like Nonoxynol-9; phenoxypolyethoxylethanols like nonyl phenoxypolyethoxylethanol and octyl phenoxypolyethoxylethanol; glucoside alkyl ethers like octyl glucopyranoside; maltoside alkyl ethers like dodecyl maltopyranoside; thioglucoside alkyl ethers like heptyl thioglucopyranoside; digitonins; glycerol alkyl esters like glyceryl laurate; alkyl aryl polyether sulfates; alcohol sulfonates; sorbitan alkyl esters; cocamide ethanolamines like cocamide monoethanolamine and cocamide diethanolamine; sucrose monolaurate; dodecyl dimethylamine oxide, N,N-Dimethylmyristylamin-N-oxid (Deviron®) and sodium cholate.

Detergents particularly useful in the method disclosed herein include non-ionic surfactants like polyoxeethylene glycol sorbitan alkyl esters, including polysorbates such as Polysorbate 20 (such as Tween 200), Polysorbate 40 (such as Tween) 40°, Polysorbate 60 (such as Tween 60°), and Polysorbate 80 (such as Tween 80°); and polyoxyethylene octyl phenyl ether (such as Triton® X-100). The terms “Polysorbate” and “Tween” as used herein are interchangeable. The concentration of the detergent is equal to or greater than about 0.1% (w/w) and equal to or less than about 2% (w/w); in particular the concentration of the detergent is equal to or greater than 0.2% (w/w) and equal to or less than 1.5% (w/w). In a more specific embodiment of the present invention, the concentration of the detergent is equal to or greater than 0.75% (w/w) and equal to or less than 1.25% (w/w). More specifically, the concentration of the detergent is selected from the group consisting of: about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.2% (w/w), about 1.5% (w/w), about 1.7% (w/w), or about 2% (w/w). More in particular, the concentration the organic solvent is at least about 0.2% (w/w), at least about 0.5% (w/w), at least about 0.75% (w/w), at least about 1% (w/w), or at least about 1.25% (w/w). In certain preferred embodiments, the concentration of the detergent is about 0.75% (w/w), or about 1% (w/w), or about 1.25% (w/w). In a more preferred embodiment, the concentration of the detergent is about 1% (w/w). The present invention also includes the concentrations of the organic solvent at intervals of 0.05% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w) or 1% (w/w) between the above cited concentrations.

In one aspect of the present invention, the polypeptide material is incubated with a solvent, or a detergent, or a mixture of solvent and detergent for an incubation time equal to or greater than about 5 minute and equal to or less than about 120 minutes. Specifically, the incubation time is selected from the group comprising about 5 minutes, about 10 minutes, about 20 minutes, about 30 minutes, about 60 minutes, about 90 minutes and about 120 minutes. More specifically, said incubation time is at least about 5 minutes, at least about 10 minutes, at least about 20 minutes, at least about 30 minutes, at least about 60 minutes, at least about 90 minutes and at least about 120 minutes. Even more specifically, the incubation time is equal to or greater than 30 minutes and equal to or less than 90 minutes, preferably the incubation time is 60 minutes. The present invention also includes the solvent and detergent incubation time at intervals of 1, 2, 5, 10, 15, 30, 60, 75 or 90 minutes between the above cited incubation intervals.

According to one aspect of the present invention, a harvested polypeptide material is first loaded into an affinity chromatography column that has been equilibrated with an equilibration buffer and then incubated with said mixture of a solvent and a detergent within said affinity chromatography column, wherein said solvent is TnBP and said detergent is Polysorbate 80. More specifically wherein the concentration of TnBP is comprised between about 0.05% (w/w) and about 1% (w/w), and wherein the concentration of Polysorbate 80 is comprised between about 0.5% (w/w) and about 2% (w/w); preferably wherein the mixture of a solvent and a detergent comprises about 0.3% (w/w) TnBP and about 1% (w/w) Polysorbate 80.

According to another aspect of the present invention, the polypeptide material is incubated with a solvent, or a detergent, or a mixture of solvent and detergent at a pH comprised between 5.5 and 9, preferably at a pH comprised between 6.5 and 8.5, more preferably between 7 and 8. In particular, the polypeptide material is incubated with a solvent, or a detergent, or a mixture of solvent and detergent at a pH selected from the group comprising about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9; preferably at pH about 7, about 7.1, about 7.2, about 7.3, about 7.4, about 7.5, about 7.6, about 7.7, about 7.8, about 7.9, about 8, more preferably at pH 7.4 or pH 8.

Following protein A chromatography and viral inactivation treatment, or following viral inactivation treatment and protein A chromatography the solution containing the polypeptide of interest, such as an antibody, may be further treated by other chromatography and filtration steps to further purify and concentrate the antibody for producing a bulk drug substance containing the therapeutic protein. For instance, the once the polypeptide is eluted from the PA column the polypeptide containing solution may undergo one or more steps of ion exchange chromatography (such as cation exchange chromatography and/or anion exchange chromatography and/or hydrophobic interaction chromatography and/or mixed mode chromatography), and one or more steps of filtration, such as ultrafiltration/diafiltration, virus nanofiltration. The purified polypeptide may further be subjected to step of excipients addition and concentration for the production of the bulk drug substance.

Viral inactivation assays allow to test the purity of the antibody solution and assure viral safety. In a preferred embodiment of this invention the antibody-containing solution is spiked with a model virus and the viral presence in the samples is measured before and after spiking. Model viruses include but are not limited to murine leukemia virus (MLV), Murine Minute Virus (MMV), Pseudorabies Virus (PRV).

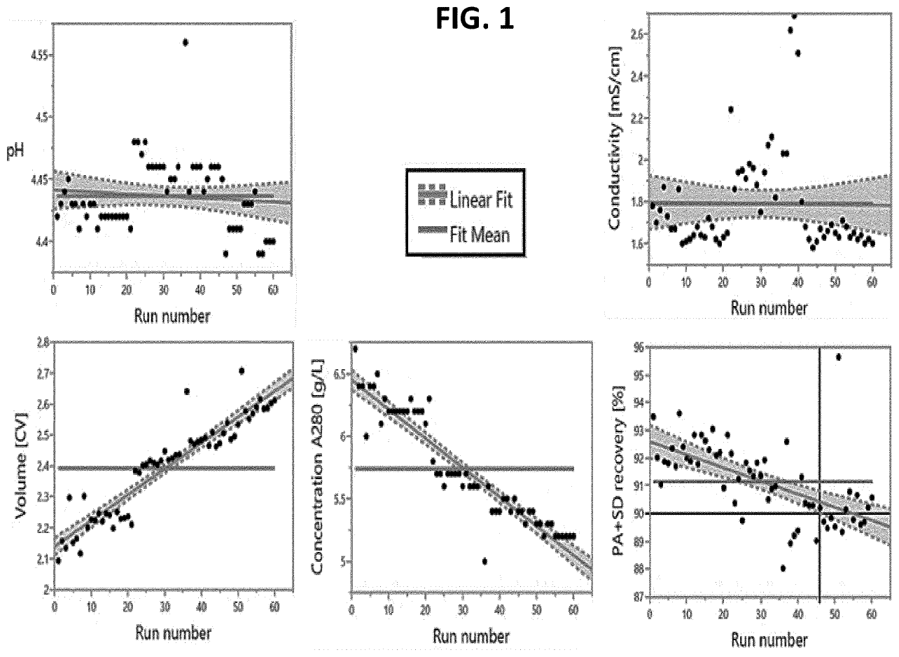

FIG. 1: Correlation analysis between performance attributes and the number of runs performed.

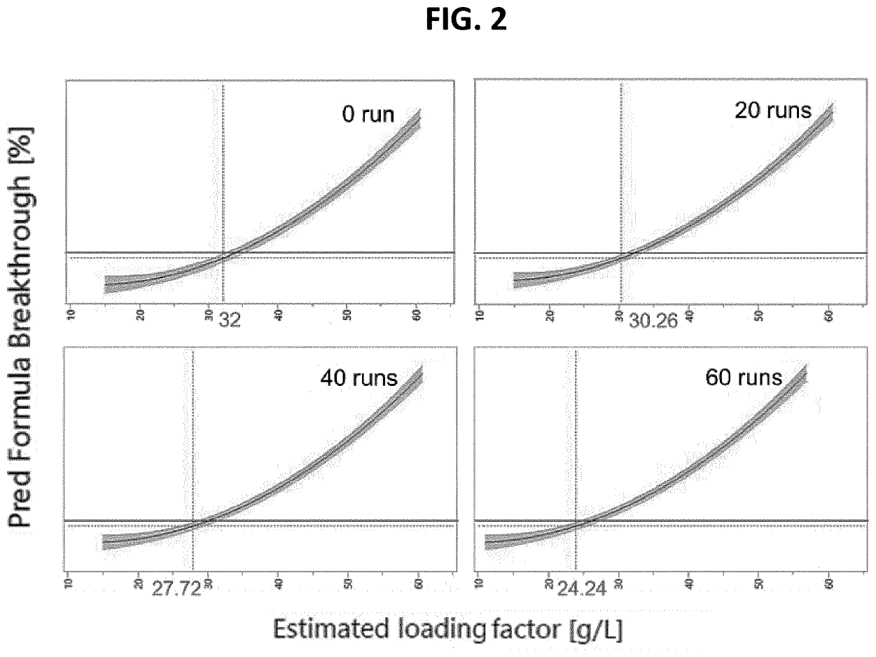

FIG. 2: DBC calculation and progress throughout the study.

### EXAMPLES

### Example 1: Methods for Inactivating Viral Contaminants

### Materials

The starting materials used in this study were two clarified harvests generated by cell culture and clarification processes. Both clarified harvest #A and #B have been produced with the same Chinese Hamster Ovarian (CHO) cell line, respectively at a 3 L development scale and at a 250 L pilot scale. The antibody is a BEAT® bispecific antibody: BEAT #1.

The list of the chemicals and buffers used is presented in the Table 1.

TABLE 1
Buffers and chemicals used during the study

| Process step | Item | |
|---|---|---|
| VI by solvent/ | Solvent | Tri-n-butyl phosphate (Merck) |
| detergent (SD) | Detergent | Polysorbate 80 (Merck) |
| Protein A | HETP equilibration | 0.1M NaCl |
| (PA) | HETP spike | 2M NaCl |
| | Equilibration | PBS pH 7.4 |
| | S/D buffer post | PBS pH 7.4 + 1.0% (w/w) PS 80 + |
| | loading | 0.3% (w/w) TnBP |
| | Wash 1 + S/D | 0.1M Tris, 1M NaCl pH 8.0 + |
| | | 1.0% (w/w)PS 80 + 0.3% |
| | | (w/w)TnBP |
| | Wash 1 | 0.1M Tris, 1M NaCl pH 8.0 |
| | Wash 2 | 0.17M Acetate pH 5.0 |
| | Elution | 0.05M Acetate pH 4.1 |
| | Strip | 0.1M Glycine pH 3.5 |
| | CIP | 0.1M NaOH |
| | Storage | 20% EtOH |

The resin used for the Protein A chromatography is KanCapA™ from Kaneka. Prepacked ValiChrom® 5.0-200 column was used for this study. Its features are summarized in Table 2.

TABLE 2
Chromatography column features

| Supplier | Column dimension | Bed height | Column volume (CV) |
|---|---|---|---|
| Repligen | 5 mm ID × | 20 cm (≥19 | 3.927 mL |
| | 200 mm H | cm-≤21 cm) | |

The equipment and instruments used throughout this study are referenced in Table 3.

TABLE 3
Laboratory equipment and instruments used during the study

| Equipment | Detailed description |
|---|---|
| Chromatography | ÄKTA Pure (Cytiva), with 2 mm UV cell |
| system | |
| Software | Unicorn ™ v.7.3. (Cytiva) |
| pH and conductivity | Mettler Toledo Seven Excellence |
| meter | |
| Bench balance | 320XT 4200C Precisa balance |
| Nanodrop | Thermo Scientific Nanodrop One |
| Filters | TPP 250 filtermax rapid |
| Vacuum pump | Merck Millipore |

### Methods

### S/D Incubation Strategies

The experiments related to the selection of the best S/D incubation strategy are performed using the clarified harvest #A. The two strategies developed herein are:
1. S/D incubation post sample loading involving the introduction of a buffer containing S/D in the Protein A process.
2. S/D incubation post sample loading and first wash.

For strategy (1) Protein A process is performed as follows:
Protein A (PA) column is equilibrated with PBS at pH 7.4 (6 CVs);
The clarified harvest is loaded into the PA column (Kaneka KanCapA™, loading factor 25 g/L);
Rinsing the PA column with 1% (w/w) Polysorbate 80 and TnBP 0.3% (w/w) in PBS at pH 7.4 (5 CVs);
Incubation with 1% (w/w) Polysorbate 80 and TnBP 0.3% (w/w) in PBS at pH 7.4 is performed in a static mode for 60 min within the PA column;
The PA column is washed with a first washing buffer comprising 0.1 M of Tris and 1 M NaCl at pH 8.0 (5 CVs);
The PA column is washed with a second washing buffer comprising 0.17 M of Acetate at pH 5.0 (5 CVs);
The antibody is eluted with an elution buffer comprising 0.05 M of Acetate at pH 4.1 (10 CVs);
The PA columns is stripped with a 0.1 M Glycine at pH 3.5 (5 CVs).

For strategy (2) Protein A process is performed as follows:
Protein A (PA) column is equilibrated with PBS at pH 7.4 (6 CVs);
The clarified harvest is loaded into the PA column (Kaneka KanCapA™, loading factor 25 g/L);
The PA column is washed with a first washing buffer comprising 0.1 M of Tris and 1 M NaCl at pH 8.0 and 1% (w/w) Polysorbate 80 and TnBP 0.3% (w/w) (5 CVs);
Incubation with 1% (w/w) Polysorbate 80 and TnBP 0.3% (w/w) is performed in a static mode for 60 min within the PA column at pH 8;
The PA column is washed with a second washing buffer comprising 0.17 M of Acetate at pH 5.0 (5 CVs);
The antibody is eluted with an elution buffer comprising 0.05 M of Acetate at pH 4.1 (10 CVs);
The PA columns is stripped with a 0.1 M Glycine at pH 3.5 (5 CVs).

The reference strategy includes S/D incubation before the Protein A chromatography step, directly in the clarified harvest, as follows:
Incubation of the clarified harvest with 1% (w/w) Polysorbate 80 and TnBP 0.3% (w/w) is for 60 to 90 min;
Protein A (PA) column is equilibrated with PBS at pH 7.4 (6 CVs);
The inactivated clarified harvest is loaded into the PA column (Kaneka KanCapA™, loading factor 25 g/L);
The PA column is washed with a first washing buffer comprising 0.1 M of Tris and 1 M NaCl at pH 8.0 (5 CVs);
The PA column is washed with a second washing buffer comprising 0.17 M of Acetate at pH 5.0 (5 CVs);
The antibody is eluted with an elution buffer comprising 0.05 M of Acetate at pH 4.1 (10 CVs);
The PA columns is stripped with a 0.1 M Glycine at pH 3.5 (5 CVs).

### Resin Lifetime

The clarified harvest used throughout the resin lifetime evaluation is the clarified harvest #B. Once the best S/D incubation strategy is selected between strategy (1) and (2), a reuse study of the Protein A resin was performed. It aims to evaluate the impact of such S/D incubation on the column performances. To do so, 60 runs were performed following the strategy previously selected.

The resin lifetime is assessed by monitoring the process performance attributes (pH, conductivity, concentration, volume and step recovery) and the product quality (monomer and BEAT % and TnBP levels). In addition, the Dynamic Binding Capacity (DBC) at 10% breakthrough will be determined at the beginning of the study and then, every 20 runs.

The DBC determination consists in loading more product than can be bound on the column (overload). To do so, 70 g of BEAT #1 were loaded per liter of resin. The DBC is identified at 10% breakthrough, meaning when only 90% of the loaded material binds on the column. It is determined by fractionating the PA flowthrough and quantifying the BEAT #1 concentration in these fractions.

### S/D Stability Study

A S/D stability study will be conducted. This stability is assessed by quantifying the TnBP and the PS 80 within the buffer used for the selected strategy, on the preparation day (T₀) and five weeks later (T₅). All samples are held at room temperature and protected from light up to the sample collection meaning when the time point is achieved. They are then stored at −20° C. until the analysis.

### Analytical Methods

Size Exclusion High Pressure Liquid Chromatography (SE-HPLC) is used to determine the percentage of BEAT #1 monomers, by separating them from aggregates and fragments. The intact BEATs are distinguished from the fragmented ones by Non Reduced Capillary Gel Electrophoresis (CGE-NR or non-reduced CE-SDS). The BEAT percentage in each PA eluates are compared to the proportion in the clarified harvest, to assess the ability of the run to purify the starting material.

Before both a SE-HPLC and a non-reduced CE-SDS, clarified harvest samples are purified through a PA spin-trap chromatography to eliminate most of the components coming from the cell-culture.

BEAT concentration in the clarified harvest used as PA load is determined by Protein A High Pressure Liquid Chromatography (PA-HPLC). The BEAT concentration in the PA eluate is assessed by determining the sample absorbance at 280 nm.

In addition, the levels of TnBP and PS 80 are quantified in the PA intermediates (i.e. load, flowthrough, wash 1 fraction, wash 2 fraction and eluate). Polysorbate 80 levels are assessed by UV-spectrophotometry, and TnBP quantification is performed by Gas Chromatography-Mass Spectrometry (GC-MS).

Finally, as one of the objectives of the PA step is the removal of the Host Cell Proteins (HCP) present in the clarified harvest, the level of these impurities is measured in the PA eluates coming from the tested strategies. The HCP quantification is performed with the 3G Elisa HCP kit of Cygnus technologies which covers about 50% of CHO cells HCP.

### Results and Discussion

### S/D Incubation Strategy Selection

The strategy selection is mainly based on the ability of the Protein A to remove the solvent and the detergent introduced for the viral inactivation S/D mix (TnBP and polysorbate 80). However, additional product quality (HCP level and BEAT %) and performance attributes (pH, conductivity, concentration, volume and yield) are also monitored to be compared to the reference run.

### Performance Attributes and Product Quality

All the PA runs were performed at similar loading factors (i.e., amount of product loaded per liter of resin). The outputs considered for the selection of the S/D incubation strategy are summarized in Table 4, except for the TnBP and PS 80 levels which will be discussed in the next paragraph.

TABLE 4
Performance attributes and product quality
of the strategy selection runs

| | | PA Eluate | | |
|---|---|---|---|---|
| | Clarified | S/D incubation | | |
| | Harvest | | Strategy | Strategy |
| | #A | Reference | (1) | (2) |
| pH | 6.9 | 4.8 | 4.8 | 4.8 |
| Conductivity [mS/cm] | 15.4 | 4.4 | 4.2 | 4.2 |
| Concentration [g/L] | 0.647 | 7.8 | 7.5 | 7.3 |
| Volume [CV] | — | 3.6 | 3.7 | 3.8 |
| Loading factor (g/L) | — | 28 | 28 | 28 |
| HCP conc. [ppm or ng/mg] | 1039232 | 575 | 567 | 305 |
| HCP removal [%] | — | 99.94 | 99.95 | 99.97 |
| Monomer (SE-HPLC) [%] | 89 | 97 | 96 | 97 |
| BEAT (CGE NR) [%] | 81 | 83 | 83 | 83 |
| PA + SD recovery [%] | — | 99 | 101 | 100 |

All performance attributes and product quality from the three runs performed revealed similar. Whatever the S/D incubation strategy, no significant differences were observed neither between the PA eluate features (i.e., pH, conductivity, concentration, volume and yield), nor with regards to the product quality (i.e., monomer and BEAT %, HCP level). Moreover, resulting chromatogram profiles were comparable.

### Solvent/Detergent Levels

PS80 and TnBP are process related impurities introduced for the viral inactivation. They have to be removed and remain at traces level (i.e., below 1 ppm) at the end of the downstream process. As it is known that the PA is the relevant step to remove these impurities, especially the TnBP, the goal is to reach this traces level in the PA eluate. Therefore, concentrations of both chemicals were measured in the S/D matrix (inactivated clarified harvest or S/D buffer depending on the strategy), as a control, as well as in the PA eluate.

Table 5 confirms that expected TnBP and PS80 concentrations were loaded on the column during the three runs performed. In addition, regardless of the viral inactivation, the results showed that no PS 80 remained in the PA eluate. On the contrary, while only traces of TnBP were detected in the PA eluates resulting from the S/D incubation in the clarified harvest and post sample loading, an S/D incubation post wash 1 led to a final TnBP level of 8 ppm which was higher than the industrial standards (<1 ppm).

TABLE 5
PS 80 and TnBP levels of the strategy selection runs

| | | S/D incubation | | | | | | |
|---|---|---|---|---|---|---|---|---|
| | | Reference | | Strategy (1) | | Strategy (2) | | |
| | | PA load | PA eluate | SD buffer | PA eluate | SD buffer | PA eluate | |
| PS 80 | Effective | 1.0% | <0.0025% | 1.0% | <0.0025% | 1.0% | <0.0025% | |
| level | Expected | 1.0% | — | 1.0% | — | 1.0% | — | |
| TnBP level | Effective | 0.3% | <1 ppm | 0.3% | <1 ppm | 0.3% | 8 | ppm |
| | Expected | 0.3% | <1 ppm | 0.3% | <1 ppm | 0.3% | <1 | ppm |

Although bot strategies (1) and (2) lead to surprising and encouraging results, the viral inactivation strategy selected for further studies was the first, namely S/D incubation post loading on the Protein A. The reuse study was therefore performed following the S/D incubation post sample loading process.

### Resin Lifetime Assessment

The reuse study was performed following the S/D incubation strategy previously selected i.e. post sample loading incubation. Although 60 runs were planned to be performed during this study, some additional runs have to be considered (DBC runs and S/D incubation strategy selection). At the end of the project, the resin had undergone 70 runs.

### Performance Attributes

Firstly, in order to assess the resin lifetime, performance attributes of the PA eluate were monitored along with the runs (Table 6) and then compared using JMP® software for making correlations between each output and the number of runs performed (FIG. 1).

TABLE 6
Performance attributes monitored from the resin lifetime evaluation

| | PA eluate | | | | | | |
|---|---|---|---|---|---|---|---|
| | Run 1 | Run 10 | Run 20 | Run 30 | Run 40 | Run 50 | Run 60 |
| pH | 4.4 | 4.4 | 4.4 | 4.5 | 4.5 | 4.4 | 4.4 |
| Conductivity [mS/cm] | 1.8 | 1.6 | 1.6 | 1.8 | 2.5 | 1.6 | 1.6 |
| Concentration A280 [g/L] | 6.7 | 6.2 | 6.1 | 5.6 | 5.4 | 5.3 | 5.2 |
| Volume [CV] | 2.1 | 2.2 | 2.2 | 2.5 | 2.5 | 2.5 | 2.6 |
| Loading factor (g/L) | 15 | 15 | 15 | 15 | 15 | 15 | 15 |
| PA + SD recovery [%] | 94 | 92 | 91 | 91 | 89 | 90 | 91 |

For each attribute, the correlation analysis consists in comparing the fit mean to the linear fit, considering the related 95% confidence interval. pH and conductivity means are included in this confidence interval therefore, these outputs can be considered stable throughout the study. PA+SD recovery as well as PA eluate volume and titer were significantly correlated to the cycle number. A significant decrease in the concentration was noticed whereas the volume increased overtime, leading to a slight decline in the recovery. This progressive dilution of the PA eluate was likely to be attributed to the column compression. Indeed, from the first to the last run, the bed height dropped from 20.0 to 19.2 cm. In addition, such a compression has been already observed with these small devices over cycles which did not involve S/D. Therefore, despite these differences, all the process performances are within acceptable ranges.

### Product Quality

Table 7 shows the evolution of monomer and BEAT percentages throughout the cycles.

TABLE 7
Quality attributes monitored from the resin lifetime evaluation

| | PA eluate | | | | | | |
|---|---|---|---|---|---|---|---|
| | Run 1 | Run 10 | Run 20 | Run 30 | Run 40 | Run 50 | Run 60 |
| PS80 level | Pending | — | Pending | — | Pending | — | Pending |
| TnBP level [ppm] | <1 | — | <1 | — | <1 | — | <1 |
| Monomer (SE-HPLC) [%] | 97 | — | 97 | — | 99 | — | 98 |
| BEAT (CGE NR) [%] | 87 | — | 92 | — | 87 | — | 89 |

Based on these results, it appeared that the use of S/D incubation within the column had no impact neither on the product quality, nor on the TnBP removal. Indeed, monomer and BEAT proportions and TnBP concentration remained constant throughout the study, respectively around 98%, 90% and at traces level.

### Dynamic Binding Capacity (DBC) Evolution

FIG. 2 depicts the results of the DBC definitions performed at the beginning of the study and every 20 runs afterwards. Models were calculated from the data, by JMP®, to predict the 10% breakthrough.

These results show a decrease in the DBC, from 32 to 24 g_{BEAT #1}/L of resin, meaning a 25% decline. Therefore, the more runs were performed on the column, the lower the capacity of the resin was.

The selected S/D incubation strategy showed similar performances throughout the reuse study, in terms of performance attributes (pH, conductivity) and product quality (BEAT % and TnBP level). The slight variations observed with regards to the concentration, volume and yield were more probably attributed to the cycle repetition and not to the S/D incubation within the column.

Regarding the DBC at 10% breakthrough, the 25% decrease observed over 60 runs was comparable to what indicated by the supplier (according to Kaneka, there should be 20% decrease in the DBC 5% after 300 PA runs). Consequently, it can be deduced that the S/D incubation within the column may have an impact on the binding capacity and thus, on the resin lifetime. However, a DBC decrease may only impact the process yield but not the process and product related performances.

### Solvent/Detergent Mix Stability

The levels of TnBP measured in the buffer are summarized in the Table 8.

TABLE 8
TnBP levels for S/D stability study

| Time point | T₀ (Preparation date) | T₅ (After 5 weeks) |
|---|---|---|
| TnBP concentration [%] | 0.3 | 0.3 |

These results confirm that there was no degradation of the TnBP in these storage conditions. PS 80 it is known to be stable for several months at room temperature as it is typically used as excipient for drug substance formulation. Therefore, the buffer comprising PBS pH 7.4+1.0% (w/w) Polysorbate 80+0.3% (w/w) TnBP can be considered stable during 5 weeks at room temperature (21° C.) and protected from light.

### Example 2: Virus Inactivation Study

A virus inactivation study was performed as follows for the virus inactivation strategy (1).

### Materials

Xenotropic Murine leukaemia virus (MLV) was chosen as relevant model virus for a virus inactivation study. MLV represents a non-defective gamma retrovirus. Inclusion of MLV is mandatory for biological products derived from CHO cell lines and monoclonal antibody products (see guidelines ICHQ5A, CHMP/BWP/398498/2005 and CPMP/BWP/268/95). The characteristics of MLV are summarized in Table 9.

TABLE 9
Viruses to be used in clearance study

| | | | | | | Resistance to physical/ |
|---|---|---|---|---|---|---|
| Virus | Strain | Genome | Envelope | Family | Size (nm) | chemical reagents |
| MLV | pNFS Th-1 | ssRNA | Yes | Retro | 80-110 | Low |

The study was performed on BEAT #1 clarified harvest. The process buffers and chemicals are listed in Table 10.

TABLE 10
Process buffers and chemicals used in clearance study

| | Item description | Intended to be used for (step) |
|---|---|---|
| Chemicals | Polysorbate 80 | Chemical inactivation |
| | Tri-n-butyl phosphate | |
| Buffers | PBS pH 7.4 | Protein A affinity |
| | PBS pH 7.4 + S/D | |

### Methods

### Samples Preparation

The Protein A load material was thawed in a water bath at 20° C. to 25° C., with gentle inversion and the container will be removed from the water bath as soon as the ice has completely melted. Then it was 0.22 μm filtered after thawing and equilibrated to process temperature/room temperature (i.e., +20±5° C.).

### Virus Preparation

MLV was sonicated on full power for 1 minute per 1 ml volume in a cup horn sonicator or 2×4 minutes in a sonicating water bath (with 1 minute rest between sonications) prior to use.

### Study Design and Process Steps

Generation of fresh material (Solvent/Detergent treated resin slurry where proteins were bound to the resin beads), for the Chemical Inactivation (CI) step, needed to be performed. To do so, a “partial” Kaneka KanCapA Affinity Chromatography was performed as a single run at 15° C. to 25° C. The column was firstly equilibrated using PBS pH 7.4 (6 CV), approximately 705 mL of BEAT #1 clarified harvest were loaded (i.e., proteins bound to the resin beads by affinity) and to finish, the resin was washed with at least 5 CV of PBS pH 7.4+S/D (i.e., PBS pH 7.4, 1.0% (w/w) Polysorbate 80, 0.3% (w/w) TnBP).

The resin has been then unpacked using the aforementioned buffer (to push) and the resulting S/D treated resin slurry (also containing proteins bound to the resin beads) has been used to perform the subsequent chemical inactivation (CI) step. This step was conducted as a single run with kinetic inactivation investigated over 60 minutes as this is the hold time in the Protein A column.

The CI experimental design consisted in spiking MLV virus into the S/D treated resin bulk, mixing for 2 minutes before collecting samples at different time points (0-1, 15, 30 and 60 minutes). All samples were 0.45 μm filtered to remove any resin and quenched at a dilution selected as a result of the pre-study testing. A TCID₅₀ infectivity assay was then performed on each sample to provide the viruses content.

### Results and Conclusions

The virus inactivation obtained was ≥5.41+0.39 Log₁₀, which corresponds to an effective contribution to the viral clearance (see for reference Table 11, showing viruses removal effectiveness, based on the guidelines indicated above, e.g., ICHQ5A, CHMP/BWP/398498/2005 and CPMP/BWP/268/95).

TABLE 11
Viruses removal effectiveness

| Removal/inactivation contribution | Log₁₀ reduction factor |
|---|---|
| Effective | ≥4 Log₁₀ |
| Moderately effective | ≥2 Log₁₀-<4 Log₁₀ |
| Contributing to the removal of viruses | ≥1 Log₁₀-<2 Log₁₀ |
| Ineffective | <1 Log₁₀ |

1. A method for preparing a virus-inactivated polypeptide solution, comprising the steps of (i) harvesting a polypeptide material produced by transfected cells comprising the coding sequences of said polypeptide, which have undergone cell culture, (ii) viral inactivation treatment upon said harvested polypeptide material consisting of incubation with a mixture of a solvent and a detergent, characterized in that said harvested polypeptide material is first loaded into an affinity chromatography column that has been equilibrated with an equilibration buffer and then incubated with said mixture of a solvent and a detergent within said affinity chromatography column.

2. The method of claim 1, wherein said affinity chromatography column is a Protein A affinity chromatography column.

3. The method of claim 1 or claim 2, wherein the incubation of said harvested polypeptide material with said mixture of a solvent and a detergent is performed at a pH comprised between about 6.5 and about 8.5.

4. The method of any one of the preceding claims, wherein the incubation of said harvested polypeptide material with said mixture of a solvent and a detergent is preceded by at least one washing step.

5. The method of any one of the preceding claims, wherein the incubation of said harvested polypeptide material with said mixture of a solvent and a detergent is followed by at least one washing step.

6. The method of any one of the preceding claims, wherein said polypeptide is eluted from said affinity chromatography column with an elution buffer.

7. The method of any one of the preceding claims, wherein said solvent is TnBP and said detergent is Polysorbate 80.

8. The method of any one of the preceding claims, wherein said mixture of a solvent and a detergent comprises TnBP at a concentration comprised between about 0.05% (w/w) and about 1% (w/w), and Polysorbate 80 at a concentration comprised between about 0.5% (w/w) and about 2% (w/w).

9. The method of any one of the preceding claims, wherein said mixture of a solvent and a detergent comprises about 0.3% (w/w) TnBP and about 1% (w/w) Polysorbate 80.

10. The method of claim 4, wherein said washing step that precedes the incubation of said polypeptide with said mixture of a solvent and a detergent is performed with a washing buffer comprising about 0.1 M of Tris buffer and 1 M NaCl at pH 8.0 and 1% (w/w) Polysorbate 80 and TnBP 0.3% (w/w).

11. The method of claim 5, wherein said at least one washing step that follows the incubation of said polypeptide with said mixture of a solvent and a detergent is performed with a washing buffer that comprises about 0.17 M of Acetate at pH 5.0.

12. The method of claim 5, wherein the incubation of said harvested polypeptide material with said mixture of a solvent and a detergent is followed by two washing steps and wherein the first of said two washing steps is performed with a washing buffer comprising about 0.1 M of Tris buffer and 1 M NaCl at pH 8.0, and the second of said two washing steps is performed with a washing buffer comprising about 0.17 M of Acetate at pH 5.0.

13. The method of any one of the preceding claims comprising the steps of:
(a) Equilibrating a protein A affinity chromatography column with an equilibration buffer comprising PBS at pH 7.4;
(b) Loading a polypeptide material onto said Protein A affinity chromatography column material at a loading factor 25 g/L;
(c) Incubating said polypeptide material with the mixture of a solvent and a detergent of claim 9 for about 60 minutes;
(d) Subjecting said protein A affinity chromatography column to two washing steps according to claim 12;
(e) Eluting said polypeptide with an elution buffer comprising about 0.05 M Acetate at pH 4.1;

14. The method of any one of the preceding claims wherein said polypeptide is an antibody or an antibody fragment thereof.

15. The method of claim 14 wherein said antibody or antibody fragment thereof is multispecific.
